SEQUENCE LISTING (9 sequences)

SEQ ID NO: 1 - DNA, 33 bp, Artificial Sequence
  Seq. ID No. 1 Forward Primer MLH1-qMSPJHF1
cgatagattaggtatagggttttatcgtttttc

SEQ ID NO: 2 - DNA, 29 bp, Artificial Sequence
  Seq. ID No. 2 Reverse Primer MLH1-qMSPJHR1&2
gcccaaaaaaaacaaaataaaaatcgacg

SEQ ID NO: 3 - DNA, 33 bp, Artificial Sequence
  Seq. ID No. 3 Forward Primer MLH1-qMSPJHF2
ttattattaaataacgttgggtttattcgggtc

SEQ ID NO: 4 - DNA, 24 bp, Artificial Sequence
  Seq. ID No. 4 Probe MLH1-qMSPJHP
acgttgggtttattcgggtcggaa

SEQ ID NO: 5 - DNA, 34 bp, Artificial Sequence
  Seq. ID No. 5 Forward Primer MLH1-qMSPJHF3
cgatagattaggtatagggttttatcgtttttcg

SEQ ID NO: 6 - DNA, 35 bp, Artificial Sequence
  Seq. ID No. 6 Forward Primer MLH1-qMSPJHF4
cgatagattaggtatagggttttatcgtttttcgg

SEQ ID NO: 7 - DNA, 33 bp, Artificial Sequence
  Seq. ID No. 7 Forward Primer MLH1-qMSPJHF5
gatagattaggtatagggttttatcgtttttcg

SEQ ID NO: 8 - DNA, 21 bp, Artificial Sequence
  Seq. ID No. 8 Forward Primer MLH1-qMSPP3F1
ttttgcgggaggttataagag

SEQ ID NO: 9 - DNA, 20 bp, Artificial Sequence
  Seq. ID No. 9 Reverse Primer MLH1-qMSPP3R1
cgcttctcaaactcctcctc

(19) United States
(12) Patent Application Publication
(10) Pub. No.: US 2018/0223367 A1
(43) Pub. Date: Aug. 9, 2018

(54) ASSAYS, METHODS AND COMPOSITIONS FOR DIAGNOSING CANCER

(71) Applicants: Jim Z. Lu, Buffalo Grove, IL (US); Zhongmin Guo, Buffalo Grove, IL (US)

(72) Inventors: Jim Z. Lu, Buffalo Grove, IL (US); Zhongmin Guo, Buffalo Grove, IL (US)

(73) Assignee: GoPath Global, LLC, Indianapolis, IN (US)

(21) Appl. No.: 15/364,592

(22) Filed: Nov. 30, 2016

Related U.S. Application Data
(62) Division of application No. 14/194,276, filed on Feb. 28, 2014, now abandoned.
(60) Provisional application No. 61/771,061, filed on Feb. 28, 2013.

(51) Int. Cl. C12Q 1/6886

(52) U.S. Cl. CPC C12Q 1/6886; C12Q 2600/112

(57) ABSTRACT

The present invention provides a method and single-tube assay for identification and quantitative analysis of differentially methylated MLH1 promoter sequences that are associated with certain types of cancer in an individual by obtaining a biological sample comprising DNA from the individual, detecting the presence of and measuring the level of methylated MLH1 promoter sequences, and comparing the presence of and level of methylation in the sample to a normalization reference of “normal” beta-actin gene promoters, wherein a difference in the level or pattern of MLH1 methylation of the sample compared to the Actin gene reference level identifies abnormally methylated MLH1 promoter sequences associated with cancer.

This application is a divisional of U.S. application Ser. No. 14/194,276, filed Feb. 28, 2014, which claims the benefit of U.S. Provisional Application No. 61/771,061, filed Feb. 28, 2013, which are hereby incorporated by reference in their entirety.

### SEQUENCE LISTING

The instant application contains a Sequence Listing, which has been submitted via EFS-Web as an ASCII copy named 335390003_ST25.txt, 2,236 bytes in size, created on Apr. 10, 2014, and which is hereby incorporated by reference in its entirety.

### BACKGROUND OF THE INVENTION

The present invention relates to conditions characterized by differentially methylated MLH1 promoter sequences and, in particular, to diagnostic and prognostic methods that exploit the presence of such DNA sequences that exhibit altered MLH1 promoter sequences.

In one embodiment, the invention may be used as a diagnostic for certain cancers and non-cancerous neoplastic diseases having hypermethylation of the MLH1 promoter, including but not limited to colorectal disease, endometrial carcinoma and gastric cancer.

For example, Lynch syndrome (LS) is a hereditary form of colorectal cancer (CRC) and is responsible for 2-5% of newly diagnosed patients with CRC. LS is caused by germline mutations in DNA mismatch repair (MMR) genes (MLH1, MSH2, MSH6, PMS2), which lead to high microsatellite instability (MSI-H) and loss of MMR protein expression. However, only about 15% of MSI-H CRC is associated with LS and the remaining 85% are largely sporadic in origin, whereby mismatch repair deficiency is caused by promoter hypermethylation silencing of the MLH1 gene, and this is often combined with BRAF mutation. Therefore, MLH1 promoter DNA methylation combined with BRAF V600E mutation has been accepted as a reliable and standard prior art molecular test to differentiate LS and sporadic CRC with the MSI-H phenotype and to identify LS-related CRC patients needing genetic testing. The characteristics of LS as a hereditary colon cancer syndrome and its association with these genetic markers are described in H. F. A. Vasen et al. (2007) J Med. Genet. 44:353-362, M. Gala et al. (2011) Semin. Oncol. 38:490-499, R. S. Nelson et al. (2009) Curr. Oncol. Rep. 11:482-489, E. Lastra et al. (2012) Clin. Transl. Oncol. 14:254-262, E. Domingo et al. (2004) J. Med. Genet. 41:664-668 and E. Domingo et al. (2005) Oncogene 24:3995-3998, which are incorporated herein by reference in their entirety.

LS is but one of the multiple types of cancer and non-cancerous neoplastic disease which is within the scope of the present invention. Other cancers may include endometrial carcinoma and gastric cancers, which are also within the scope of the invention, as well as any future discovered cancer or other neoplastic disease which exhibits hypermethylation of the MLH1 gene promoter.

Several prior art methods exist to determine MLH1 DNA methylation status tissues. However, these methods either are non-quantitative, or they use primers and probes not detecting exclusively methylated MLH1 DNA, or primers and probes not selectively targeting the promoter genomic region critical for MLH1 expression. Such methods are described in M. Bettstetter (2007) Clin. Cancer Res. 13:3221-3228, C. A. Eads et al. (2000) Nucl. Acids Res. 28:e32, K. Rand et al. (2002) Methods 27:114-120, H. Thomassin (2004) Nucl. Acids Res. 32:e168 and G. Deng et al. (1999) Cancer Res. 59:2029-2023, which are incorporated herein by reference in their entirety. Moreover, none of these methods has been verified in large patient cohorts, as discussed in M. T. Parsons et al. (2012) J. Med. Genet. 49:151-157, which is incorporated herein in its entirety. These limitations prevent scientists and clinicians from rendering an unambiguous interpretation of the MLH1 methylation status of tested tumors.

Two highly cited methods for analysis of MLH1 methylation are described in M. Bettstetter et al. (2007) and C. A. Eads et al. (2000). However, we have found that neither method provides a consistently clear cut-off for unambiguous determination of MLH1 methylation in a group of CRC tumors with known information for MLH1 protein expression and BRAF mutations.

Conventionally, combined analyses of MSI, MMR protein expression, MLH1 promoter methylation and BRAF mutation have been considered a standard molecular test for selecting LS candidates for further genetic testing. E. Lastra et al. (2012). However, the currently available methods for MLH1 DNA methylation frequently generate inconsistent results among different studies and therefore cannot be applied for diagnostic purposes. A comparison of different strategies for MLH1 methylation is described in L. Perez-Carbonell (2010) J Mol. Diagn. 12:498-504, which is incorporated herein by reference in its entirety. Furthermore, there is no FDA-approved standard test for MLH1 methylation in clinical use today.

Additionally, the selection of patients for genetic testing to diagnose LS is challenging in clinical practice. The Amsterdam and Bethesda criteria for identifying individuals who should be tested for MSI have substantial limits in sensitivity and specificity for LS detection, as described in S. A. Wahlberg et al. (2002) Cancer Res. 62:3485-3492 and A. Umar (2004) J Natl Cancer Inst. 96:261-268, which are incorporated herein by reference in their entirety. As a result, a significant number of LS patients are overlooked and many patients without mismatch repair gene mutations are sent for genetic testing, leading to an unnecessary increase in inconvenience for the patients and in laboratory cost. Therefore, additional, more specific testing means are needed.

A conventional test including a combined analyses of MSI, MMR protein expression, MLH1 promoter methylation and BRAF mutation have been considered a standard molecular test for selecting LS candidates for further genetic testing. However, the currently available methods either are non-quantitative, or they use primers and probes not detecting exclusively methylated MLH1 DNA, or primers and probes not selectively targeting the promoter genomic region critical for MLH1 expression. These limitations prevent an unambiguous interpretation of the MLH1 methylation status of tested tumors and result in false positives.

Thus, a need exists for a reproducible method for unambiguous detection of certain neoplastic cells in patients including but not limited to those containing colorectal, gastric and endometrial cancers, and quantitative measurement of DNA methylation in MLH1 promoter DNA, that provides a discrete measure of positive versus negative DNA methylation. A need also exists for a method of continuous measure of levels and patterns of DNA methylation to classify and predict different types and stages of cancer, cancer therapeutic outcomes and patient survival.

### SUMMARY OF THE INVENTION

A method and single-tube assay are disclosed for identification and quantitative analysis of differentially methylated MLH1 promoter DNA sequences that are associated with some cancers and neoplastic diseases in general in an individual by obtaining a biological sample comprising DNA from the individual, detecting the presence of and measuring the level of methylated MLH1 promoter sequences, and comparing the presence of and level of methylation in the sample to a normalization reference level of “normal” beta-actin gene promoters which is amplified in the same single-tube reaction, wherein a difference in the level or pattern of methylation of the sample compared to the normalization reference level identifies abnormally methylated MLH1 promoter sequences associated with such cancers and other neoplastic diseases, including but not limited to some CRCs, endometrial cancers and gastric cancers. In a further embodiment, a single-tube assay for determining the presence of neoplastic disease in a subject is disclosed, the assay comprising: isolating a single-stranded DNA encoding MLH1 from a biological sample taken from the subject using the probe of the invention, wherein the biological sample is selected from tissue, urine, stool, saliva, blood and serum; treating the single-stranded DNA with bisulfite; amplifying the DNA using the primers of the invention, and determining the level of methylation of the MLH1 promoter region of the single stranded DNA, wherein the presence of MLH1 promoter methylation is an indication of the presence of neoplastic disease in the subject. In an alternative embodiment, the method and single-tube assay can be combined with a miniaturized array platform that allows for a high level of assay multiplexing and scalable automation for sample handling and data processing. Genomic probe and corresponding primers are also disclosed, that are useful in the methods of the invention as they enable detection of differentially methylated genomic MLH1 promoter sequences currently associated with colorectal cancers, endometrial carcinoma and gastric cancer although it may be associated with other cancers and other phenotypes in the future.

### BRIEF DESCRIPTION OF THE DRAWINGS

FIG. 1 is a graphic representation of locations of primers and probes for the MLH1 promoter.

FIG. 2A is a data plot of the fluorescence intensity and number of cycles for quantitative methylation specific PCR, using prior art primers for the MLH1 promoter.

FIG. 2B is a data plot of the fluorescence intensity and number of cycles for quantitative methylation specific PCR, using primers that flank at least a portion of the region of the MLH1 promoter sequence from −248 to −178 bp relative to the transcription start site.

FIG. 2C is a data plot of the fluorescence intensity and number of cycles for quantitative methylation specific PCR, using primers that flank at least a portion of the region of the MLH1 promoter sequence from −248 to −178 bp relative to the transcription start site.

FIG. 3A is a data plot of the methylation percentage of CRC tumors negative for BRAF mutation (Group 1) and MSI-H, MLH1 protein negative and BRAF mutation positive (Group 2), as determined by quantitative methylation specific PCR using prior art primers.

FIG. 3B is a data plot of the methylation index (Mdex) of CRC tumors negative for BRAF mutation (Group 1) and MSI-H, MLH1 protein negative and BRAF mutation positive (Group 2), as determined by quantitative methylation specific PCR using primers that flank at least a portion of the region of the MLH1 promoter sequence from −248 to −178 bp relative to the transcription start site.

FIG. 4 shows the sequences of embodiments of forward and reverse oligonucleotide primers that flank all or a portion of the region of the MLH1 promoter sequence from −248 to −178 bp relative to the transcription start site, and the sequence of an oligonucleotide probe that is complementary to that region.

### DETAILED DESCRIPTION OF THE INVENTION

Assays, compositions and methods are disclosed for the accurate and sensitive detection of differential methylation of genomic MLH1 promoter DNA in clinical samples. These assays, compositions and methods are useful to enable diagnostic and prognostic methods for conditions that are characterized by a level and/or pattern of methylated genomic MLH1 promoter DNA distinct from the level and/or pattern of methylated genomic MLH1 promoter DNA exhibited in the absence of the particular condition.

The quantitative detection of only methylated MLH1 DNA sequences is enabled by the use of novel primers and probes that target the genomic region essential for MLH1 protein expression. In particular, a panel of nucleic acid primers and one probe are disclosed that are useful for the detection of differentially methylated genomic MLH1 promoter DNA that can be correlated to the presence of or susceptibility to neoplastic disease, including CRC, endometrial carcinoma and gastric cancer in an individual with MMR gene deficiency. The unique design of primers and probes as well as an in-tube normalization control in the assay provide an accurate and sensitive test for MLH1 DNA methylation. In light of the high incidence of applicable cancers (including but not limited to colorectal, gastric and endometrial), and perhaps other non-cancerous neoplastic diseases, and the requirement to test a large percent of patients for MLH1 methylation, these methods should significantly improve the effectiveness of clinical care of cancer patients.

Although described in detail with reference to various types of CRC, it is further anticipated that the disclosed assays, compositions and methods can be utilized in any neoplastic disease or condition in which methylation of the MLH1 promoter occurs. Such conditions may include but are not limited to CRC, endometrial and gastric cancers, and may further include other cancers and non-cancerous neoplastic conditions. The disclosed assays, compositions and methods are also useful for predicting the susceptibility of an individual to a condition that is characterized by a level and/or pattern of methylated genomic MLH1 promoter DNA sequences that is distinct from the level and/or pattern of methylated genomic MLH1 DNA sequences exhibited in the absence of the condition.

Because methylation detection targets genomic DNA, rather than RNA or protein, it offers several technological advantages in a clinical diagnostic setting: (1) readily available source materials, particularly important for prognostic research, because typically DNA can be more reliably extracted than RNA from archived biological samples for study; (2) capability for multiplexing, allowing simultaneous measurement of multiple targets to improve assay specificity; (3) easy amplification of assay products to achieve high sensitivity; and (4) the ability to detect a positive signal in neoplastic cells that arises from methylation inactivation of at least one allele of the mismatch repair genes.

The diagnostic and prognostic assay for CRC (or other diseases) is performed by methylation-specific polymerase chain reaction (PCR) of the MLH1 promoter in the critical region from −248 to −178 bp relative to the transcription start site, or a portion of this critical region. Sample genomic DNA is analyzed by treatment with sodium bisulfite, as is known in the art. Bisulfite treatment converts the cytosine residues of the DNA to uracil, but does not modify methylated cytosines (5-methylcytosine). PCR of the bisulfite treated DNA sample is performed using a pair of forward and reverse primers that flank at least a portion of the region of the MLH1 promoter sequence from −248 to −178 bp relative to the transcription start site. One or both of the forward and reverse primers is complementary to at least a portion of this region and overlaps one or more methylation sites within the critical region. Such primers will only anneal to and amplify methylated sequences that are resistant to bisulfite conversion. Sensitivity of the assay is increased using primers that overlap multiple methylation sites and/or where the methylation site is at the 3′ end of the primer. In an alternative embodiment, those of skill in the art will appreciate that a converse assay may also be performed using unmethylated-specific primers.

In a preferred embodiment, the assay is performed by quantitative, real time PCR. The amplification of methylated MLH1 promoter DNA may be detected by various means known in the art. For example, a double-stranded DNA binding, fluorescent reporter dye may be used, such as SYBR® Green (Life Technologies—Grand Island, N.Y.). The amplification of DNA product during PCR is detected and measured by the increase in fluorescence intensity. The degree of amplification may be quantified relative to a standard DNA sample, and may also be normalized relative to non-methylation specific amplification.

In a particularly preferred embodiment, the amplification of methylated MLH1 promoter sequences is detected and quantified using a fluorescent reporter probe, as is known in the art. The TaqMan® Assay (Life Technologies) is an exemplary real time PCR system using fluorescent reporter probes. An oligonucleotide probe that is complementary and hybridizes to the amplified MLH1 promoter DNA is labeled with a fluorescent reporter at the 5′ end and a quencher of fluorescence at the 3′ end. PCR is performed using a polymerase that has 5′ to 3′ exonuclease activity, such as the Taq polymerase. During PCR of the MLH1 promoter DNA, polymerization proceeds until it reaches the oligonucleotide probe, where the exonuclease activity cleaves the fluorescent reporter from the 5′ end of the oligonucleotide probe, separating the fluorescent reporter from the quencher, and allowing the detection of unquenched fluorescence. The amplification of the MLH1 promoter sequences produces a proportional increase in fluorescence. Those of skill in the art will appreciate that the process may be multiplexed by amplification and detection of multiple sequences using different colored fluorescent probes.

In an alternative embodiment, the assay also provides a control for amplification of non-methylated sequences. Conventional quantitative, real-time methylation specific PCR generally uses a two-tube PCR system in which the methylated target gene and a non-methylated normalization gene are amplified in two independent PCR reactions. This experimental system sometimes may create a significant amplification bias between the reaction of the target gene and that of the normalization control—e.g., owing to differential amplification efficiency in different PCR reactions and variations in sample pipetting.

To minimize this experimental bias, the assay may comprise a single-tube quantitative, real-time methylation specific PCR assay that detects methylation of the MLH1 critical region and a normalization gene in the same reaction tube. The assay contains all components (e.g., primers and probes) for quantitative PCR amplification of the MLH1 target and the normalization gene, except for test sample DNA. The probes for detection of the methylation of MLH1 and for the normalization gene are labeled by different reporter dyes. Exemplary normalization genes include the beta-actin gene (ACTB), and the reaction conditions and PCR components are well known to those of skill in the art.

The assay provides a number of advantages that make it an accurate molecular test for MLH1 DNA methylation as compared to the prior art. First, the primers and probes in our assay are designed to amplify exclusively methylated MLH1 DNA and to target specifically the MLH1 promoter region critical for its expression. These features of the assay assure a reliable interpretation of MLH1 DNA methylation which best correlates with genuine MLH1 methylation status and expression of MLH1 protein. Second, the combined amplification of both MLH1 methylation and the normalization ACTB gene in single-tube reaction mitigates the effects of technical bias resulting from independent amplification of MLH1 methylation and the control ACTB template.

In contrast, the prior art methods for determining MLH1 DNA methylation in CRC patients were developed for research purposes. These methods either are non-quantitative, or use the primers and probes not detecting exclusively methylated MLH1 DNA, or primers and probes not selectively targeting the promoter genomic region critical for MLH1 expression. Moreover, none of these methods has been verified in large patient cohorts.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### Example 1

### Primers and Probe

Various embodiments are disclosed that enable the identification of reliable MLH1 methylation markers for the improved diagnosis and prediction of the susceptibility, diagnosis and staging of neoplastic disease, including CRC. To develop a reliable assay for accurately detecting MLH1 DNA methylation, a novel quantitative real-time system with primers and probe was designed for amplifying exclusively methylated MLH1 DNA. These primers and probe specifically target the region of the MLH1 promoter region critical for its expression, as identified in G. Deng et al. (1999). As discussed in detail below, the assay has been found to provide an accurate determination of MLH1 methylation status in CRC tissue.

Although several methods have been developed for MLH1 DNA methylation, these are mainly for research purposes and none of them has been successfully developed into an accepted standard assay for clinical molecular diagnostic use. A comprehensive study has identified the DNA sequence from −248 to −178 bp in MLH1 promoter sequences as a genomic region tightly correlated with MLH1 expression in CRC cell lines and tumors. However, the primers and probes from many previous methods do not specifically target this critical region and thus the detected MLH1 methylation by these methods may not consistently correlate with MLH1 protein expression in CRC tumors. Moreover, our testing has disclosed that primers from some highly-cited prior-art methods are not highly selective in detecting methylated MLH1 DNA. To overcome the above potential issues in previously reported assays, we designed a set of primers and probes specifically targeting the region of the MLH1 promoter from −248 to −178 bp relative to the transcription start site, which are useful for the exclusive detection of methylated MLH1 sequences.

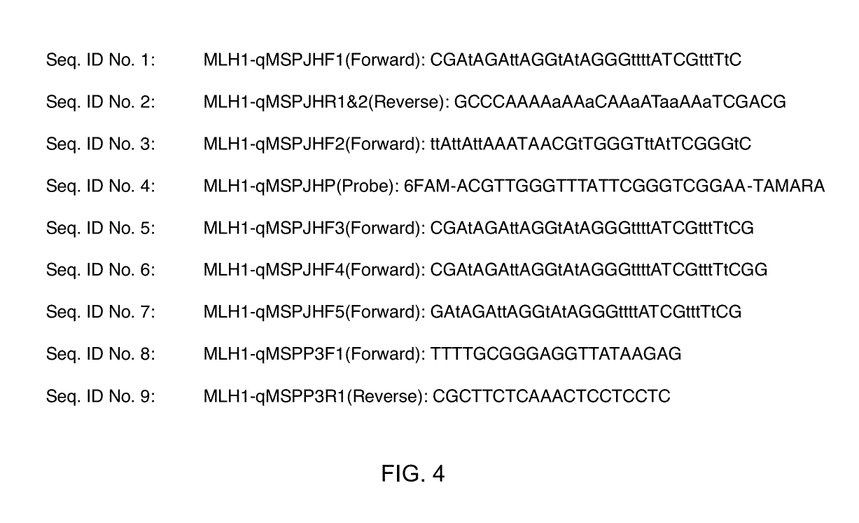

Referring to FIG. 4, reliable genomic sequences are disclosed for the detection of genomic targets for use in the diagnostic and prognostic methods described herein, which have been designated as Seq. ID Nos. 1-9, wherein Seq. ID Nos. 1, 3 and 5-8 are forward oligonucleotide primers, Seq. ID Nos. 2 and 9 are reverse oligonucleotide primers, and Seq. ID No. 4 is an oligonucleotide probe. These primers and the probe correspond to the region of the MLH1 promoter from −248 to −178 bp relative to the transcription start site and are used to detect differential methylation of genomic MLH1 promoter sequences that serve as markers associated with certain neoplastic diseases. Nucleotides shown in lower case indicate that the nucleotide corresponds to the most common nucleotide in the consensus sequence.

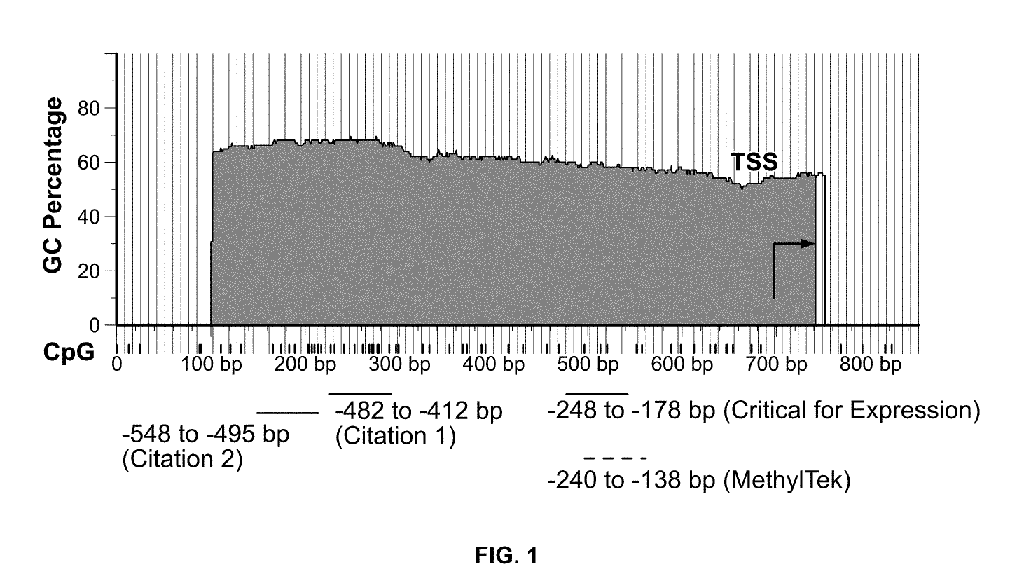

The location of these primer sequences (MethylTek) is shown in the genomic map of FIG. 1, which also indicates the MLH1 promoter methylation sites (CpG) and target region (critical for expression) from −248 to −178 bp relative to the transcription start site. Also shown are the locations of the prior art primers disclosed in Bettstetter et al. (2007) (citation 1) and Eads et al. (2000) (citation 2). TSS denotes the transcription starting site.

It is understood that the genomic target sequences provides the context for the one or more selected genomic MLH1 promoter sequences being measured within a particular genomic target sequence. Furthermore, any fraction of the total genomic MLH1 dinucleotide sequences within a genomic target sequence can be measured, including one or more, two or more three or more, four or more, five or more or all of the genomic MLH1 dinucleotide sequences within a genomic target sequence. Although FIG. 4 sets forth a particular nucleic acid probe that corresponds to the known genomic targets of the MLH1 promoter region, this probe combined with one or more primers as shown in FIG. 4 produces a surprisingly robust and unambiguous means of identifying hypermethylated MLH1 promoter DNA.

The nucleic acid probe and amplification primers are capable of detecting hypermethylated regions within the known genomic target of the MLH1 promoter region and can be employed to detect altered levels of methylation of genomic MLH1 promoter sequences in a biological sample compared to a reference level.

Any combination of these forward and reverse primers may be used. Exemplary combinations include, Seq. ID Nos. 1+2+4, Seq. ID Nos. 2+3+4, Seq. ID Nos. 2+5+4, Seq. ID Nos. 2+6+4, Seq. ID Nos. 2+7+4, and Seq. ID Nos. 8+9+4. Other useful combinations of the probe with various primers are within the scope of the present invention.

### Example 2

### Assay for Detecting MLH1 Methylation

The assay for detecting MLH1 DNA methylation combines amplification of MLH1 methylation and ACTB normalization control in a novel one-tube system. This design minimizes the amplification bias between MLH1 and the ACTB control due to variations from pipetting and amplification efficiency in different PCR reactions. The assay comprises the primers and 6-FAM/TAMRA probe for MLH1 methylation selected from those set forth in FIG. 4 (although other probes and primers may be further developed), as well as primers and probes for the ACTB control. The probes for detection of MLH1 methylation and the ACTB control are labeled with different reporter dyes—e.g., 6-FAM and HEX, respectively. However, any suitable reporters now known or hereafter developed is within the scope of the invention. Exemplary primers for the ACTB control are known in the art. For example, VIC/TAMRA labeled probes are commercially available from Applied Biosystems (ABI) and Life Technologies.

Sample genomic DNA was prepared and treated with sodium bisulfite, as is known in the art. The bisulfite treated sample DNA was then mixed with the following components: 200 μM of each dNTP, 0.5-1 μM of each primer, 0.2 μM of each probe, 1 unit of Taq DNA polymerase (AmpliTaq-Gold®-ABI, Life Technologies), and 2.0-4.0 mM of magnesium chloride. PCR was performed using the ABI 3900 and Roche 480 quantitative real-time PCR systems. PCR reaction conditions and cycling parameters were used as generally suggested by the manufacturers. Those of skill in the art will appreciate that the amounts of the various reaction components are merely examples, and a range of suitable amounts of each of the reagents may be used. Furthermore, the reaction is not limited to the use of Taq polymerase, and any polymerase suitable for use in PCR and having 5′ to 3′ exonuclease activity, now known or hereafter developed, is within the scope of the invention. In addition, other suitable PCR systems are commercially available.

### Example 3

### Test Quantitation

The assay was tested for the ability to selectively detect methylated DNA. The assay was performed as described in Example 2, using the forward primer MLH1-qMSPJHF1 (Seq. ID No. 1), the reverse primer MLH1-qMSPJHR1&2 (Seq. ID No. 2), and the probe MLH1-qMSPJHP (Seq. ID No. 4), as shown in FIG. 4. In vitro methylated lymphocyte DNA was used as a positive control. Non-methylated or non-bisulfite treated lymphocyte DNA were used as negative controls.

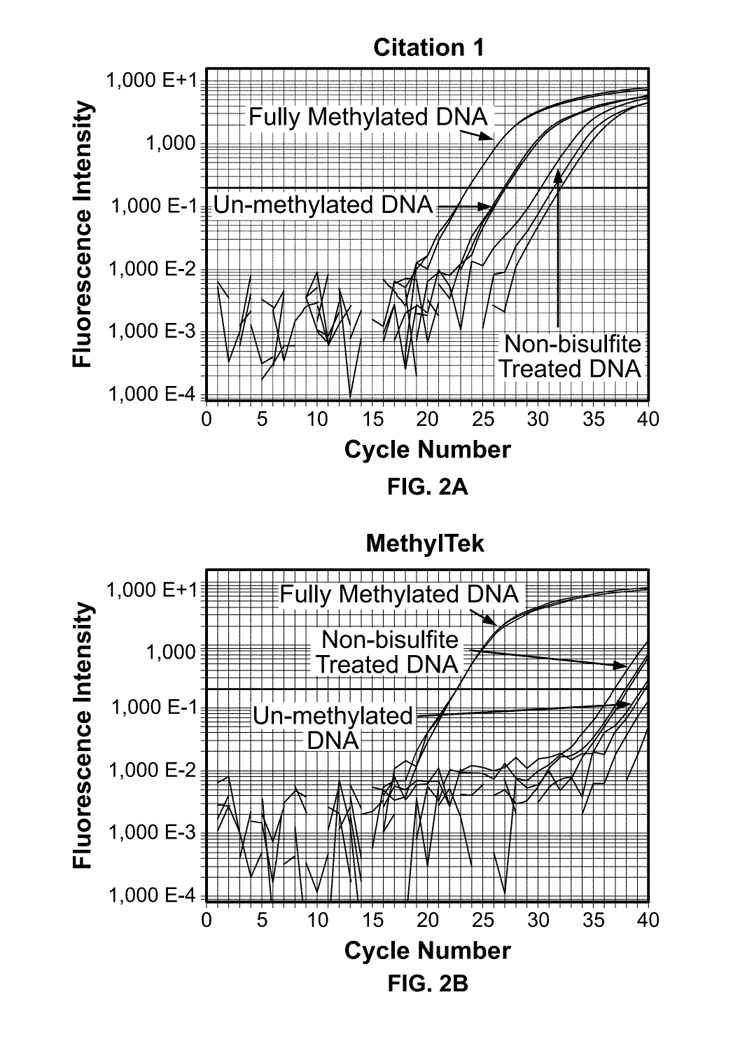

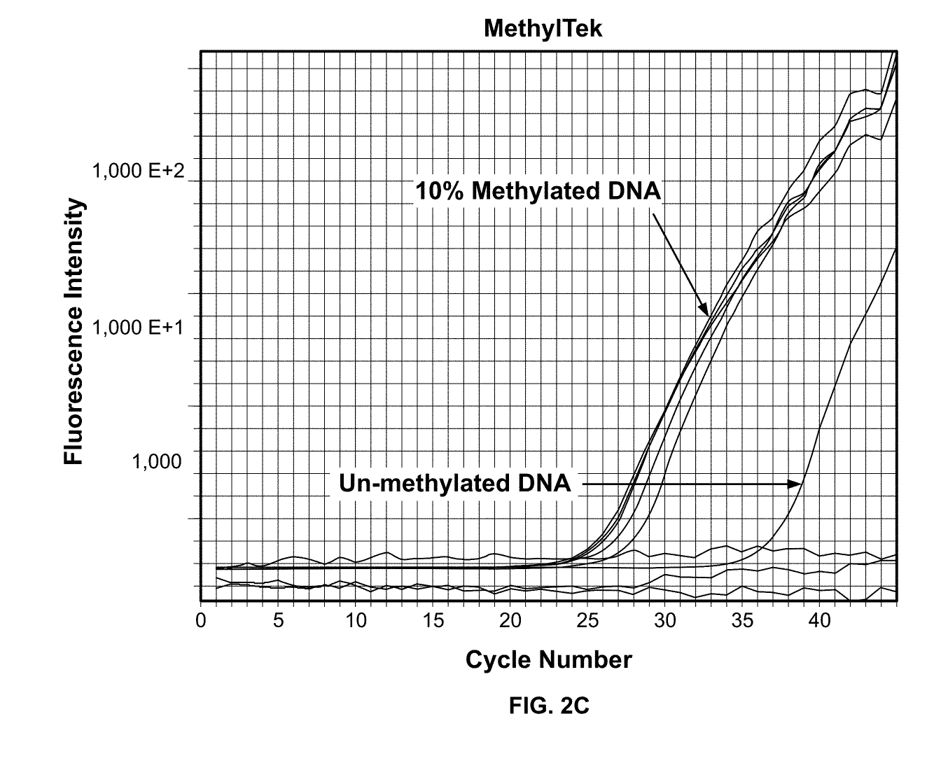

The assay was found to be highly specific and sensitive in detecting MLH1 DNA methylation in comparison to the highly cited, prior art method of Bettstetter (2007) (FIG. 1). As shown in FIG. 2A, the method of Bettstetter (2007) had only 8-fold difference (3 cycles) in selectively detecting in vitro fully methylated DNA over non-methylated DNA. Moreover, this method was also found to nonspecifically amplify bisulfite unconverted DNA.

In contrast to the prior art methods, the present assay showed more than 1000-fold selectivity (>13 cycles) in detecting methylated DNA as compared to un-methylated DNA and with no trail of amplification of bisulfite untreated DNA, as shown in FIG. 2B. Further serial dilution experiments showed that the assay can detect 10% of methylated DNA in a mixture of 90% of un-methylated DNA with close to 1000-fold selectivity (about a 10 cycle difference) over completely un-methylated DNA, as shown in FIG. 2C.

### Example 4

### Analysis of Biological DNA Samples

The assay was tested for the ability to selectively detect methylated DNA in patient tissues, and to determine an unambiguous and reliable cut-off for detection of MLH1 methylation. MLH1 methylation in 41 CRC tumors was analyzed using the assay as described in Example 2 and compared to the prior art method of Bettstetter (2007) (FIG. 1). The CRC tumors were divided into two groups. Group 1 tumors were negative for BRAF mutation and, therefore, are negative for MLH1 DNA methylation. Group 2 tumors were MSI-H, MLH1 protein negative and positive for BRAF mutation and, therefore, should bear somatic MLH1 promoter hypermethylation.

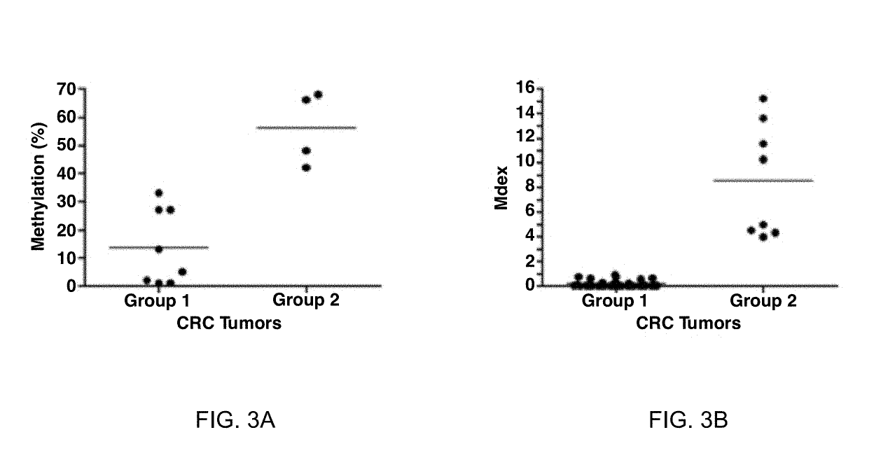

The CRC tumors were analyzed for methylated MLH1 DNA using prior art methods of Bettstetter et al. (2007) (FIG. 1), as shown in FIG. 3A. The methylation percentage was calculated as disclosed in Bettstetter et al. (2007). A high level of MLH1 methylation was detected in Group 2 tumors. However, the prior art method also detected a relatively high level of MLH1 methylation in some Group 1 tumors with positive MLH1 protein expression. Thus, the prior art method exhibited non-specific amplification of unmethylated or partially-methylated MLH1 DNA sequences, or detection of the methylated sequence in promoter region irrelevant to MLH1 expression.

In contrast to the prior art, analysis of the CRC tumors using the present assay provided an unambiguous and accurate interpretation of MLH1 DNA methylation, as shown in FIG. 3B. To prove the efficacy of the assay in primary CRC tumors, the 41 CRC tumors were assessed for MLH1 DNA methylation by the assay in a blinded fashion. A comprehensive analysis of MSI, MMR protein expression and BRAF V600E mutation was performed. MMR protein expression was analyzed by immunohistochemistry, MSI by fragment analysis, BRAF mutation by sequencing and MLH1 DNA methylation by the assay of the invention in over 500 CRC tissue samples.

The methylation index (Mdex) was calculated according to the formula:

Mdex=2^{-(CT of MLH1-CT of ACTB)}×10

where CT is the number of PCR cycles. The optimal cut-off for MLH1 promoter methylation was determined according to the status of MLH1 protein expression, MSI and BRAF mutation, and further confirmed by bisulfite DNA sequencing. Performance (sensitivity and specificity) of the assay was evaluated by appropriate statistical analysis.

Surprisingly, all 8 tumors with positive MLH1 methylation (Mdex>3) were MSI-H, MLH1 protein negative, and BRAF mutation positive (FIG. 3B, Group 2), while the remaining 33 tumors (FIG. 3B, Group 1) were MLH1 methylation negative (Mdex<1) and none of them harbored BRAF mutation. The prevalence of MLH1 methylation between two groups was highly significant (p value<0.0001, Fisher's exact test, two-sided). No case fell into the ambiguous Mdex range between values 1 to 3 (FIG. 3B).

These experiments verify that the assay is effective as a clinical diagnostic assay for MLH1 DNA hypermethylation in cancer patients, specifically CRC patients. Analysis of the 41 CRC tumors generated a Mdex cut-off zone of 1-3, rather than a single cut-off value for interpretation of methylation as in the prior art Bettstetter (2007). Tumors negative for MLH1 expression and BRAF mutation inevitably had Mdex values approximately above 3 while all tumors with positive MLH1 expression or from LS patients had Mdex below 1. No tumor had an ambiguous Mdex value within the gray zone of 1-3.

The assay of the invention is presumed to work on other neoplastic tissues evidencing hypermethylation of MLH1, although the Mdex values may be somewhat different. Use of the assay to diagnose other types of neoplastic disease (such as but not limited to gastric cancers and endometrial cancer) are considered within the scope of the invention.

Although the invention has been described in detail with reference to preferred embodiments, variations and modifications exist within the scope and spirit of the invention as described and defined in the following claims.

What is claimed is:

1. A method for analysis of a DNA sample for the methylation status of the MLH1 promoter associated with neoplastic disease, comprising the steps of:
providing a first pair of forward and reverse oligonucleotide primers that flank a target region of the sample DNA including at least a portion of the region of the MLH1 promoter from −248 to −178 bp relative to the transcription start site, and wherein at least one of the forward and reverse oligonucleotide primers overlaps a methylation site therein;
providing a first oligonucleotide probe that is complementary to the target region, and that is labeled with a first fluorescent reporter at the 5′ end and a quencher of fluorescence at the 3′ end
treating the DNA sample with sodium bisulfite;
mixing the sodium bisulfite treated DNA sample with the first pair of oligonucleotide primers and the first oligonucleotide probe;
amplifying the sodium bisulfite treated DNA sample mixture by polymerase chain reaction using a DNA polymerase having 5′ to 3′ exonuclease activity; and
measuring the fluorescence of the amplified DNA sample.

2. The method of claim 1, wherein the forward oligonucleotide primer is selected from the group consisting of: Seq. ID No. 1, Seq. ID No. 3, Seq. ID No. 5, Seq. ID No. 6, Seq. ID No. 7 and Seq. ID No. 8.

3. The method of claim 1, wherein the reverse oligonucleotide primer is selected from the group consisting of: Seq. ID No. 2 and Seq. ID No. 9.

4. The method of claim 1, wherein:
the forward oligonucleotide primer is selected from the group consisting of: Seq. ID No. 1, Seq. ID No. 3, Seq. ID No. 5, Seq. ID No. 6, Seq. ID No. 7 and Seq. ID No. 8;
the reverse oligonucleotide primer is selected from the group consisting of: Seq. ID No. 2 and Seq. ID No. 9; and
the oligonucleotide probe has the sequence of Seq. ID No. 4.

5. The method of claim 1, further comprising the step of extracting the DNA sample from a biological sample selected from the group consisting of: tissue, urine, stool, saliva, blood and serum.

6. The method of claim 1, further comprising the steps of:
providing a providing a second pair of forward and reverse oligonucleotide primers that flank a non-methylated region of the DNA sample;
providing a second oligonucleotide probe that is complementary to the non-methylated region of the DNA sample, and that is labeled with a second fluorescent reporter at the 5′ end and a quencher of fluorescence at the 3′ end, wherein the first and second fluorescent reporters are different; and
wherein the sodium bisulfite treated DNA sample is mixed with the first and second pair of oligonucleotide primers and the first and second oligonucleotide probes in a single tube.

7. The method of claim 6, wherein the non-methylated region of the sample DNA includes at least a portion of the beta-actin gene.

8. The method of claim 6, wherein a DNA sample having a methylated MLH1 promoter has a methylation index of about 3 or greater.
